(19) United States
(12) Patent Application Publication
(10) Pub. No.: US 2013/0203623 A1
(43) Pub. Date: Aug. 8, 2013

(54) METHOD AND KIT FOR CLASSIFYING A PATIENT

(71) Applicant: Emmanuel Zachariah, Highland Park, NJ (US)

(72) Inventor: Emmanuel Zachariah, Highland Park, NJ (US)

(73) Assignee: UNIVERSITY OF MEDICINE AND DENTISTRY OF NEW JERSEY, Somerset, NJ (US)

(21) Appl. No.: 13/806,946

(22) PCT Filed: Jun. 28, 2011
(86) PCT No.: PCT/US11/42180

§ 371 Date: Mar. 20, 2013

Related U.S. Application Data
(60) Provisional application No. 61/359,723, filed on Jun. 29, 2010.

(51) Int. Cl. C12Q 1/68

(52) U.S. Cl. CPC C12Q 1/6837
USPC 506/9; 506/16

(57) ABSTRACT

Provided is a Suppressive Subtractive Hybridization-Oligonucleotide Microarray (SSH-OM) method for the prediction of treatment response for personalized medicine applications and for the prediction of cancer classes and subclasses.

### INTRODUCTION

This application claims priority to U.S. Provisional Application No. 61/359,723, filed Jun. 29, 2010, which is incorporated herein by reference.

### BACKGROUND OF THE INVENTION

It is known that individual patients respond to medical treatment differently. This variability in response is due, in part, to genetic and epigenetic differences that affect gene expression. These differences may be present in the normal host tissue, or they may be acquired by cancer cells during transformation. Such differences may affect diverse components of treatment response, including: a drug's pharmacokinetics (e.g., metabolism or transport) or pharmacodynamics (e.g., a target or modulating enzyme); host tissue sensitivity to radiation; the sensitivity of malignant cells to cytotoxic agents, including drugs and radiation; and the ability of malignant cells to invade and metastasize. Gene expression analysis provides the foundation for studying thousands of individual alterations in gene function. These alterations in mRNA expression can be considered as biomarkers. It is possible that this genomic expression profile can be used to design treatments tailored to an individual, thus maximizing the likelihood of a favorable treatment response.

Studies of the regulation of gene expression rely upon techniques for the identification and quantitation of mRNAs/transcripts coding for specific proteins. Several methods have been developed for this purpose, each offering distinct advantages and disadvantages. Subtractive cloning and microarray methods are two widely used techniques to study differentially expressed genes. PCR based subtractive cloning is a powerful technique that allows isolation and cloning of mRNAs/transcripts differentially expressed in two cell populations. Traditional Suppression Subtractive Hybridization procedures often are technically demanding and labor-intensive methods that require large amounts of mRNA, and might give rise to falsely positive and unreproducible results.

Whole genome microarray is a powerful high throughput technology for simultaneous quantitation of thousands of genes. Gene expression microarray experiments are usually performed with RNA isolated from tissues or cells, which are amplified, labeled with detectable markers and allowed to hybridize to arrays composed of gene-specific probes that represent thousands of individual genes. The greater the number of transcripts, the larger degree of hybridization, and the more the output signal. The technology highly favors the detection of high-expression transcripts, whereas the rare transcripts are masked by their low output signals that are equal to background/noise of the microarrays. In addition, it is estimated that approximately 3000 patients' samples are needed to generate stable training, microarray data set for the prediction out come in cancer (Tinker, et al. (2006) Cancer Cell 9:333-339; Ein-Dor, et al. (2006) Proc. Natl. Acad. Sci. USA 103:5923-5928).

The combination of suppression subtractive hybridization (SSH) and cDNA microarray to characterize subtracted cDNA clones has been suggested (Petroziello, et al. (2004) Oncogene 23:7734-7745; Pan, et al. (2006) BMC Genomics Oncogene 23:7734-7745). However, the methodology remains complicated for identifying differentially expressed transcripts because of the redundancy in the subtracted clones. Indeed, these conventional combination methods sacrifice the advantages of high sensitivity of SSH because of redundancy in the subtracted amplicons; 5 to 20 subtractions are required to get enriched cDNA clones.

Thus, there remains a need for simple, sensitive, cost-effective methods to predict treatment responses in cancer patients, particularly in lung cancer. Lung cancer is the most common cause of cancer mortality in the United States for both men and women, claiming 163,510 people in year 2004. Non-Small Cell Lung Cancer (NSCLC) represents approximately 80% of the cases. Despite recent advances in multi-modality therapy, the overall 5-year survival rate remains on the order of 15% in the United States. Surgery is the first choice of treatment for localized NSCLC (stage I, II and IIIA) if the patient's physical condition is appropriate. However, the result of surgical treatment remains unsatisfactory, and 35-50% of the patients will relapse within 5 years. Early identification of patients prone to relapse immediately following surgery allows the physician to target adjuvant chemotherapy to those patients for whom it is necessary. In this respect, gene expression analysis in accordance with the present invention can be useful in early identification patient prone to recurrence.

One of the most significant advances in NSCLC research has been the demonstration of longer survival with adjuvant chemotherapy (ACT) for early-stage resected NSCLC. However, the effect of ACT on prolonging overall and disease-free survival is modest, with 4% to 15% improvement in 5 years survival, and often ACT is associated serious adverse effect (Sangha, et al. (2010) The Oncologist 15:862-872). Therefore, identifying the sub-group(s) of patients who will most likely benefit from any or a specific type of ACT would be of substantial clinical benefit.

### SUMMARY OF THE INVENTION

The present invention features a method and kit for classifying a patient. The method of the invention involves the steps of:

(a) isolating a total RNA sample from the patient;

(b) subjecting the total RNA to ribosomal RNA reduction, mRNA species enrichment and fragmentation;

(c) subtracting a first aliquot of the fragmented mRNA against a first reference pool of complementary RNA (cRNA);

(d) independently subtracting a second aliquot of the fragmented mRNA against a second reference pool of cRNA;

(e) independently amplifying the subtracted mRNA of (c) and (d) to produce first and second amplified RNAs, respectively;

(f) independently hybridizing the first and second amplified RNAs to oligonucleotide microarrays to generative first and second patterns of hybridization;

(g) comparing the first and second patterns of hybridization to controls to classify the patient.

In some embodiments of the instant method, the first and second reference pool cRNA are respectively from responders and non-responders to treatment; the patient is classified as a responder or non-responder to treatment; the controls include oligonucleotides microarray hybridization patterns of responders and non-responders to treatment and the treatment is lung cancer surgery.

In other embodiments of the instant method, the patient is classified as having a particular class of cancer such as lung cancer, which can be classified as non-small-cell lung carcinoma, small-cell lung carcinoma, cardinoid, or sarcoma, wherein the non-small-cell lung carcinoma can be further subclassified as adenocarcinoma, squamous cell carcinoma or Large Cell Carcinoma. In accordance with certain embodiments, the first and second reference pool cRNA are respectively from patients with adenocarcinoma and squamous cell carcinoma the patient is classified as having adenocarcinoma or squamous cell carcinoma; and the controls include oligonucleotides microarray hybridization patterns of patients with adenocarcinoma and squamous cell carcinoma.

In yet other embodiments, the step of comparing the first and second patterns of hybridization to controls includes analyzing and visualizing the microarray data with a Computer Aided Design (CAD)-based software.

A kit of the invention includes a first and second reference pool of complementary RNA (e.g., from responders and non-responders to treatment; or from patients with adenocarcinoma and squamous cell carcinoma); one or more oligonucleotides microarrays; and optionally includes CAD-based microarray data analysis and visualization software.

### BRIEF DESCRIPTION OF THE DRAWINGS

FIG. 1 shows a schematic SSH protocol for the preparation of disease-specific mRNA for microarray or RNA sequence analysis.

FIG. 2 shows the prediction of recurrence in a Stage 1 NSCLC patient.

FIG. 3 shows the use of a computer aided design microarray data analysis and visualization algorithm in the prediction of recurrence of lung cancer.

FIG. 4 shows the use of a computer aided design microarray data analysis and visualization algorithm for predicting adjuvant chemotherapy response in lung cancer patients.

FIG. 5 shows the use of a computer aided design microarray data analysis and visualization algorithm for predicting subclasses of lung cancer.

### DETAILED DESCRIPTION OF THE INVENTION

A simple, sensitive and cost-effective SSH-oligonucleotide microarray (SSH-OM) method for the classification of cancer, in particular lung cancer, and prediction of treatment response in cancer patients, in particular lung cancer patients, has now been developed. Tumor tissue specimens obtained from NSCLC patients were used to demonstrate the application of these methods to predict surgical treatment response after complete resection and classify NSCLC into adenocarcinoma and squamous cell carcinoma subclasses. In addition, many biomarkers that distinguished non-responder from responder of adjuvant therapy were identified.

Accordingly, the present invention provides methods for predicting responses to therapy, particular in the treatment of lung cancer and classifying cancer, especially distinguishing lung adenocarcinoma from squamous cell carcinoma. In general, the methods of the invention involve isolating a sample of total RNA from a patient and subjecting the total RNA sample to ribosomal reduction and enriching for mRNA species. Subsequently, the enriched sample is divided into two portions, a first aliquot that is subtracted against a first reference pool (e.g., a non-responder or a first class of cancer), and a second aliquot that is subtracted against a second reference pool (e.g., a responder or a second class of cancer). The subtracted first and second aliquots are then independently amplified and the resulting amplified transcripts are hybridized to a microarray containing the whole human genome. The hybridized microarray is then compared to a control to determine whether the patient will or will not respond to the therapy or determine the class of cancer.

In some embodiments, patients benefiting from the instant method include those receiving treatment for a disease or condition. In this respect, non-responders can be identified and receive an appropriate alternative therapy or adjuvant therapy. In other embodiments, patients benefiting from the instant method include those with cancer where classification can identify patients at high risk of recurrence, metastasis or those at high-risk for poor prognosis.

The methods of this invention require that a sample be taken from a patient, preferably a human patient. The sample can include a tissue or biopsy sample, such as epithelial tissue, connective tissue, muscle tissue or nervous tissue. Epithelial tissue samples include simple epithelia (i.e., squamous, cuboidal and columner epithelium), pseudo-stratified epithelia (i.e., columnar) and stratified epithelia (i.e., squamous). The connective tissue samples include embryonic connective tissue (i.e., mesenchyme and mucoid), ordinary connective tissue (i.e., loose and dense), and special connective tissue (i.e., cartilage, bone, and adipose). Muscle tissue samples include smooth (i.e., involuntary) and striated (i.e., voluntary and involuntary). Nervous tissue samples include neurons and supportive cells. In addition, the sample may contain Circulating Tumor Cells (CTC) unique to the pulmonary system, such as cells from the trachea, bronchi, bronchioli, and alveoli. Cells unique to the mouth and throat are also included such as all cell types exposed in the mouth that include cheek lining, tongue, floor and roof of the mouths, gums, throat as well as sputum samples.

Upon taking the sample from a patient, the total RNAs are isolated and extracted from the specimen. Total RNA isolation can be achieved using any of a number of well-known procedures. For example, samples are lysed in a guanidinium-based lysis buffer, optionally containing additional components to stabilize the RNA, followed by CsCl centrifugation to separate the RNA from DNA (Chirgwin et al. (1979) Biochem. 18:5294-5299). Alternatively, separation of RNA from DNA can be accomplished by organic extraction, for example, with acid phenol or phenol/chloroform/isoamyl alcohol. Alternatively, RNA may be extracted from samples based on binding of RNA to silica under chaotropic conditions. If desired, RNase inhibitors may be added to the lysis buffer. Likewise, for certain cell types, it may be desirable to add a protein denaturation/digestion step to the protocol.

Formaldehyde Fixed, Paraffin Embedded (FFPE) tissue is gold standard in tumor pathology laboratories. Isolation of RNA can be achieved using any number commercially available isolation kit such as RECOVERALL total nucleic acid isolation kit (AMBION), ARRAY GRADE FFPE RNA isolation kit (Superarray), RNEASY FFPE kit (QIAGEN) and PURELINK FFPE total RNA isolation kit (Invitrogen) or any other modified protocol.

Once RNA is extracted from the sample, the RNA is subjected to ribosomal reduction and mRNA enrichment, i.e., the removal of ribosomal and transfer RNA to enrich for mRNA species. Ribosomal reduction and mRNA enrichment can be achieved using conventional approaches, e.g., oligo (dT) column chromatography or magnetic beads coated with ribosomal probes or oligo(dT). See Sambrook, et al. (1989) Molecular Cloning-A Laboratory Manual, 2^{nd} Edition, Cold Spring Harbor Laboratory, Cold Spring Harbor, N.Y.

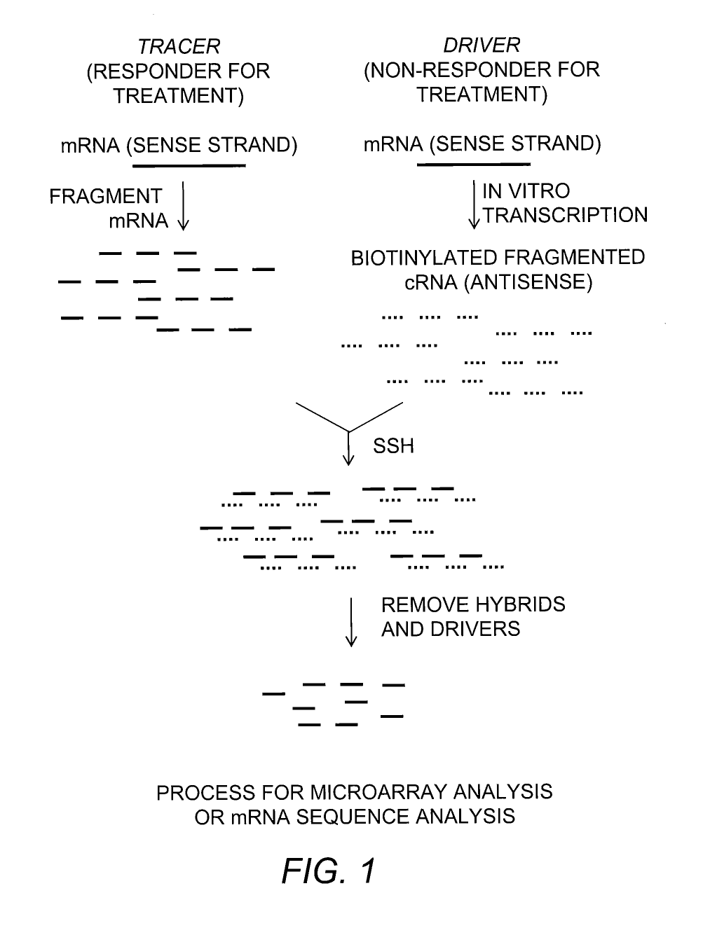

In accordance with the instant methods, the enriched mRNA sample is divided into two portions, a first aliquot that is subtracted or hybridized against a first reference pool of complementary RNA (cRNA, i.e., the antisense strand of mRNA), and a second aliquot that is subtracted against a second reference pool of cRNA. As illustrated in FIG. 1, subtraction removes RNA molecules common to both the sample and the reference. This can be achieved by, e.g., labeling of the reference cRNA with a tag or marker (e.g., biotin). The source of the reference pools will be dependent upon the analysis being conducted, e.g., determining response to treatment or classification of a patient into a class or subclass of cancer. In one embodiment, the first and second reference pools of cRNA are from responders or non-responders to a treatment, i.e., patients that respond positively to the treatment or fail respond to the treatment, respectively. In accordance with this embodiment, it is preferable that the disease or condition is cancer, particular lung cancer, and the treatment is surgical resection, adjuvant therapy, chemotherapy, targeted therapy, radiation therapy or a combination thereof. In another embodiment, the first and second reference pools of cRNA are from patients with different classes or subclasses of cancer. In accordance with this embodiment, the cancer is lung cancer which is classified into non-small-cell lung carcinoma (NSCLC), small-cell lung carcinoma (SCLC), cardinoid, or sarcoma, wherein NSCLC can be further subclassified as adenocarcinoma (AC), squamous cell carcinoma (SCC) and Large Cell Carcinoma (LCC). In this respect, the first and second reference pools of cRNA can be obtained from subjects with, e.g., NSCLC and SCLC, respectively. Alternatively, the first and second reference pools of cRNA can be obtained from subjects with, e.g., AC and SCC, respectively.

In accordance with the next step of the method, the subtracted first and second aliquots of sample mRNA are then independently amplified by conventional methods to produce first and second amplified RNAs of use in microarray analysis. For example, the mRNA can be converted to cDNA (complementary or “copy” DNA) using conventional methods and used as a template to generate cRNA by in vitro transcription. Amplification of DNA products corresponding to expressed RNA samples can also be accomplished using the polymerase chain reaction (PCR), which is described in detail in U.S. Pat. Nos. 4,683,195, 4,683,202 and 4,800,159. Alternative methods for amplifying nucleic acids corresponding to expressed RNA samples include, e.g., transcription-based amplification systems (TAS), such as that first described by Kwoh, et al. ((1989) Proc. Natl. Acad. Sci. USA 86(4):1173-7), or isothermal transcription-based systems such as 3SR (Self-Sustained Sequence Replication; Guatelli, et al. (1990) Proc. Natl. Acad. Sci. USA 87:1874-1878) or NASBA (nucleic acid sequence based amplification; Kievits, et al. (1991) J. Virol. Methods 35(3):273-86). In these methods, mRNA is copied into cDNA by a reverse transcriptase. The resulting cDNA products can then serve as templates for multiple rounds of transcription by the appropriate RNA polymerase. Transcription of the cDNA template rapidly amplifies the signal from the original target mRNA. The isothermal reactions bypass the need for denaturing cDNA strands from their RNA templates by including RNAse H to degrade RNA hybridized to DNA. Other methods using isothermal amplification, including, e.g., methods described in U.S. Pat. No. 6,251,639.

Once the first and second amplified RNAs are produced, each is independently hybridized to oligonucleotide microarrays that are representative of a genome to generative first and second patterns of hybridization. As used herein, the term “oligonucleotide microarrays that are representative of a genome” means an organized group of nucleotide sequences that are linked to a solid support, for example, a microchip or a glass slide, wherein the sequences can hybridize specifically and selectively to nucleic acid molecules expressed in a cell. The array is selected based on the organism from which the cells to be examined are derived, and, therefore, generally is representative of the genome of a eukaryotic cell, particularly a mammalian cell, and preferably a human cell. In general, an array of oligonucleotides that is “representative” of a genome will identify at least about 10% of the expressed nucleic acid molecules in a cell, generally at least about 20% or 40%, usually about 50% to 70%, particularly at least about 80% or 90%, and preferably will identify all of the expressed nucleic acid molecules. Arrays containing oligonucleotides representative of specified genomes can be prepared using well known methods, or obtained from a commercial source (e.g., AFFYMETRIX), as exemplified by the GENECHIP Human Genome HG-U133 array (AFFYMETRIX) used in the present studies. Moreover, hybridization of nucleic acids to such microarrays can be carried out by conventional protocols, typically provided by the manufacturer.

Following hybridization of the amplified RNAs to the oligonucleotide microarrays, hybridization between the amplified RNAs and the oligonucleotides of the array is detected and/or detected, and optionally quantitated. Some embodiments of the methods of the present invention enable direct detection of products. Other embodiments detect reaction products via a label associated with one or more of the amplified RNAs, e.g., a fluorescent label. In this respect, increased or decreased fluorescence intensity indicates that cells in the sample have transcribed a gene that contains the microarray oligonucleotide sequence. The intensity of the fluorescence is roughly proportional to the number of copies of a particular mRNA that were present and thus roughly indicates the activity or expression level of that gene. Arrays can paint a picture or “profile” of which genes in the genome are active in a particular cell type and under a particular condition that can be seen with the colorimetric assay.

A variety of commercially available detectors, including, e.g., optical and fluorescent detectors, optical and fluorescent microscopes, plate readers, CCD arrays, phosphorimagers, scintillation counters, phototubes, photodiodes, and the like, and software are available for digitizing, storing and analyzing a digitized video or digitized optical or other assay results, e.g., using a personal computer.

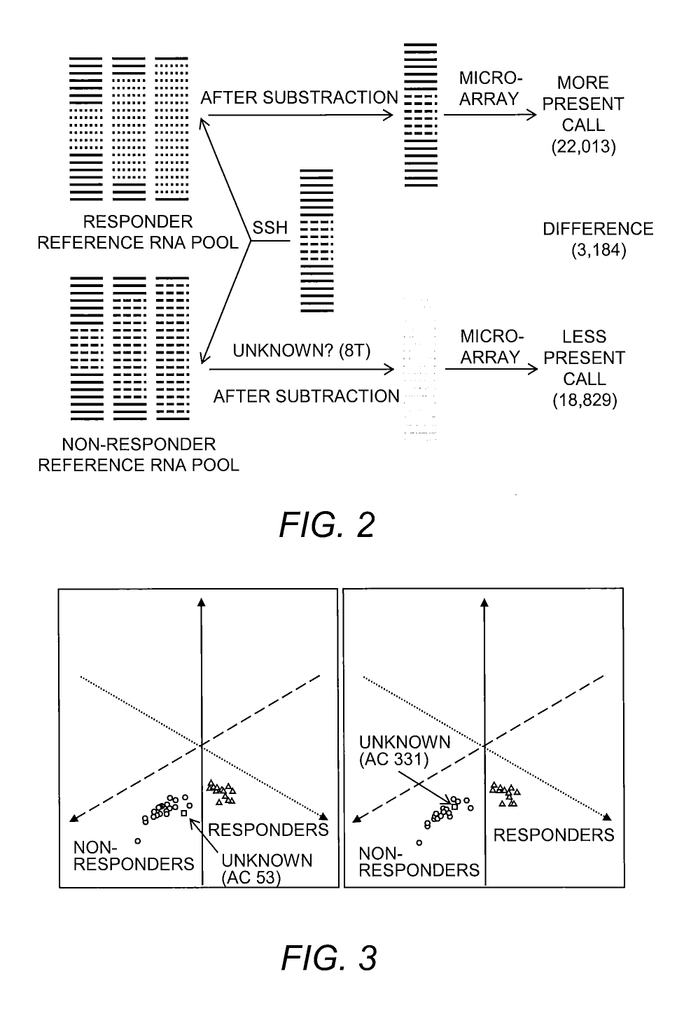

The hybridization patterns of the first and second amplified RNAs to the oligonucleotide microarrays are then compared to controls to classify the patient, e.g., as a responder or non-responder, or as having a particular class or subclass of cancer. As illustrated in FIG. 2, the more similar the patient mRNA is to a reference pool, the more the subtraction and the lesser the present call. In this respect, the patient can be readily identified by the pattern of hybridization (either qualitative, quantitative, or both) to the oligonucleotides microarray.

In an additional embodiment, the present invention provides kits for carrying out the claimed methods for analysis of gene expression. For example, a kit of the present invention can include the first and second reference pool of complementary RNA (cRNA) and one or more microarray slides (or alternative microarray format) onto which the subtracted and amplified RNA is hybridized. The kit can also include the reagents and primers suitable for use in any of the amplification methods described above. In addition, one or more materials and/or reagents required for preparing a biological sample for gene expression analysis are optionally included in the kit. Furthermore, optionally included in the kits are one or more enzymes suitable for amplifying nucleic acids, including various polymerases (RT, Taq, etc.), one or more deoxynucleotides, and buffers to provide the necessary reaction mixture for amplification. Moreover, the kit can contain instructions for carrying out each step of the claimed method.

Additionally, the kits of the present invention further include software to expedite the generation, analysis and/or storage of data, and to facilitate access to databases. The software includes logical instructions, instructions sets, or suitable computer programs that can be used in the collection, storage and/or analysis of the data. Comparative and relational analysis of the data is possible using the software provided. In particular embodiments, the software is computer aided design based microarray data analysis and visualization software.

The instant combined SSH and oligonucleotides microarray (SSH-OM) method of the invention is a simple, sensitive and cost-effective method, and it can be routinely used to predict treatment response in various other human diseases as well as in classifying cancers into classes and/or subclasses. The SSH-OM method of the invention uses small amounts of mRNA (about 100 ng), allows for direct hybridization of amplified, labeled RNAs onto whole genome oligonucleotide microarrays, takes approximately four days to complete, and transcripts generated by the SSH method are ideal for next-gen mRNA sequencing based analysis. Hence, it can be coupled with next-gen mRNA sequencing technique to analyze differentially expressed biomarkers present in various human diseases. Moreover, the method of the invention requires a small patient population (50-100) to validate prediction accuracy. Regular whole genome microarrays require more samples (˜3,000) to achieve good prediction accuracy. In addition, the method of the invention involves only one round of subtraction, where as the conventional methods require 5-20 subtractions. Furthermore, rare transcripts signals are amplified 5- to 6-fold after subtraction.

The method of the invention can be used in many applications, including, but not limited to, the prediction of treatment response, for personalized medicine applications; to predict surgical and adjuvant treatment response in lung cancer patients; to identify biomarkers present in cancer as well as other human diseases; to screen biomarkers present in various kinds of human diseases; and to classify a cancer into a class or subclass.

In this respect, some embodiments of the invention embrace the classification of a patient as having a particular class or subclass of cancer. As illustrated by the data presented in FIG. 5, the instant method was shown to correctly classify a patient with adenocarcinoma and a patient with squamous cell carcinoma based upon expression analysis using reference RNA pools from adenocarcinoma and squamous cell carcinoma patients. Cancers that can be classified by the instant invention include, but are not limited to, carcinomas (e.g., breast, prostate, lung or colon cancer); sarcoma (e.g. cancer derived from connective tissue or mesenchymal cells); lymphoma or leukemia; germ cell tumor; or blastoma. Moreover, in particular embodiments, lung cancers such as non-small-cell lung carcinoma, small-cell lung carcinoma, cardinoid, or sarcoma are classified. In addition, particular embodiments embrace the subclassification of non-small-cell lung carcinoma as adenocarcinoma or squamous cell carcinoma.

The invention is described in greater detail by the following non-limiting examples.

### Example 1

### Materials and Methods

Tissue Specimens.

Patient specimens and clinical data were obtained from Fox Chase Cancer Center, Co-Operative Human Tissue Network, NCI. The samples obtained for the present study were approved by the Internal Review Board at the UMDNJ-Robert Wood Johnson Medical School, New Brunswick, N.J.

RNA Extraction.

RNA was isolated from human tissue samples using a tissue pulverizer (Cole-Palmer). Approximately 25 mg tissue blocks were pulverized using a tissue pulverizer and total RNA was extracted using TRIZOL reagent (INVITROGEN) according to the manufacturer's instructions. The RNA was purified using the RNEASY mini kit (QIAGEN) and quality was examined with RNA 6000 Nano assay kit and the 2100 Bioanalyzer (Agilent).

Preparation of Reference RNA pool from Responder and Non-responder Patients.

Total RNA from non-responder patients was pooled to obtain “non-responder reference RNA pool.” Similarly, total RNA from responder patients was pooled to obtain “responder reference RNA pool.” Each reference RNA pool was composed of total RNA isolated from stage 1 NSCLC (adenocarcinoma) patients representing different stages of clinical spectrum. The patients were carefully chosen to ensure a broad coverage of clinical conditions. To ensure maximum coverage on human arrays, the hybridization of individual patient's RNA and the combined Reference RNA to AFFYMETRIX human whole genome HG-0133 plus 2.0 GENECHIP was evaluated. Patients RNA were chosen for the reference RNA to cover the majority of clinical conditions that were applicable for the prediction of surgical treatment response.

Suppressive Subtractive Hybridization (SSH).

Two microgram total RNA obtained from lung cancer tissue was used as the tester and driver to optimize various conditions for SSH and microarray experiments. For prediction studies, 2 μg total RNA obtained from lung cancer tissue was used as the tester, whereas 2 μg of the reference RNA pool was used as the driver. The total RNA was subjected to ribosomal reduction (INVITROGEN) to enrich mRNA species, and approximately 100 ng mRNA was fragmented and processed for SSH. Tester and driver were hybridized at various concentrations (1:10, 1:15 and 1:20), temperatures (45° C., 50° C. and 60° C.) and time intervals (4 to 24 hours) to achieve maximum specificity. A one round subtraction method was optimized for prediction studies. The unhybridized mRNA was purified using RNEASY mini elute columns according to manufacturer's recommendations (QIAGEN). The purified RNA was processed for AFFYMETRIX GENECHIP protocol.

Real Time Quantitative PCR (RT-qPCR).

Subtraction hybridization efficiencies were monitored by RT-qPCR experiments. TAQMAN assay probes for the two housekeeping genes (GAPDH and ACTB) and four cancer-related genes (CD49, EpCAM, ERBB2 and TGBR4) were purchased from Applied Biosystems (ABI). TAQMAN assays were conducted before and after SSH and the Cycle Threshold (Ct) values were correlated with SSH efficiency. TAQMAN assays were performed according to the manufacturer's instructions using MX3005P multiplex QPCR system (Agilent Technologies). The data was analyzed using MXPRO 4.1 software (Agilent Technologies).

Oligonucleotide Microarray Analysis.

The subtracted RNA was processed as recommended by AFFYMETRIX, Inc. (Santa Clara, Calif.). In brief, cDNA was synthesized from the subtracted RNA using the SUPERSCRIPT Double Stranded cDNA Synthesis kit and T7 Oligo (dT) and random primers. Using the double stranded cDNA as template, biotin labeled cRNA was generated by in vitro transcription (IVT). The cRNA was fragmented and hybridized to human whole genome HG-U133 plus 2.0 GENECHIP at 45° C. for 16 hours in an AFFYMETRIX GENECHIP Hybridization Oven 450. Each GENECHIP was then washed and stained with Streptavidin-Phycoerythrin conjugate (SAPS; Invitrogen Corp.) using an AFFYMETRIX Fluidics Station 450 and scanned on a 7G AFFYMETRIX GENECHIP scanner. Scanned images were analyzed using AFFYMETRIX GCOS 5.0 software and the output intensity files were further analyzed using the computer aided design microarray data analysis and visualization algorithm.

### Example 2

### Efficiency of SSH and Hybridization to Oligonucleotide Microarray

SSH.

A simple one round subtraction hybridization method was developed that involves in vitro transcription-based amplification to obtain biotinylated cRNA driver (FIG. 1). Initial subtractions were carried out with individual tracer mRNA against individual driver RNA to maintain the simplicity of the method. The method was composed of synthesizing biotinylated antisense RNA (cRNA) from a target tissue (Non-responder for surgical treatment: Lung cancer patient who had recurrence within 5 years after surgical resection) using in vitro transcription. The cRNA was fragmented to achieve specific hybridization kinetics. In the next step, mRNA isolated from a responder patient (Responder for surgical treatment: Lung cancer patient who did not have recurrence within 5 years after surgical resection) was hybridized with the fragmented cRNA. After hybridization, the biotinylated cRNA fragments and the hybridized targets were removed using streptavidin-coated magnetic beads. The unhybridized mRNA was tested by RT-qPCR to calculate the hybridization efficiency, and the hybridization conditions were adjusted to attain maximum subtraction efficiency.

Subtraction Efficiency.

Subtraction efficiency was calculated by RT-qPCR using TAQMAN assay probes obtained from ABI. Two housekeeping genes (ACTB and GAPDH) and four other cancer-related genes (CD49, EpCAM, ERBB2 and TGBR4) were quantitated for both responder and non-responder RNA samples. Subtraction efficiency was calculated by comparing the Ct values before and after SSH. The results are shown in Table 1.

TABLE 1

| | Responder Ct | | Non-Responder Ct | |
|---|---|---|---|---|
| Gene | Before SSH | After SSH | Before SSH | After SSH |
| ACTB | 21.57 | 29.72 | 22.80 | 27.89 |
| GAPDH | 22.00 | 28.51 | 21.40 | 28.44 |
| CD49 | 27.20 | 35.46 | 26.28 | 33.26 |
| EpCAM | 24.60 | 32.70 | 25.49 | No Ct |
| ERBB2 | 24.49 | 32.05 | 27.94 | 31.31 |
| TGFBR4 | 25.28 | 30.80 | 26.43 | 30.80 |

The Ct values for all the genes were consistently increased after SSH, indicating the specificity of the instant method. It was of particular note that one of the genes, EpCAM was completely excluded from the subtracted transcripts in the non-responder sample after SSH.

Oligonucleotide Microarray.

The unhybridized mRNA were amplified and hybridized onto AFFYMETRIX whole genome microarrays as per the manufacturer's instructions. The labeled cRNA was hybridized to human whole genome HG-U133 plus 2.0 GENECHIP. The differentially expressed transcripts were normalized using GCOS 5.0 software (AFFYMETRIX), and the output intensity files were further analyzed using computer aided design microarray data analysis and visualization algorithm. The HG-U133 Plus 2.0 microarray is composed of 1,300,000 unique oligonucleotide features covering over 47,000 transcripts and variants, which, in turn, represent approximately 39,000 of the best characterized human genes. Hence, this method is ideal to screen whole human transcriptome for the differentially expressed biomarkers.

A present call-based prediction method was developed to distinguish responders from non-responders, and vice versa. In this method, the more the similar mRNA present in a patient RNA, the more the subtraction and the lesser the present call.

Images were obtained for microarrays hybridized with RNA obtained from a responder (Patient No. 12T) without subjecting the RNA to SSH (the positive control); RNA obtained from a responder (Patient No. 12T) that was subtracted using a non-responder RNA (Patient No. 8T); and RNA obtained from a non-responder (Patient No. 8T) that was subtracted using a responder RNA (Patient No. 12T). The positive control showed approximately 58% present call for all the available 54,675 probes present on the gene chip. For RNA obtained from the responder, which was subtracted using a non-responder RNA, there was 32.4% present call for all the available 54,675 probes present on the gene chip. Approximately 14,168 probes were subtracted in this SSH experiment. For RNA obtained from a non-responder, which was subtracted using a responder RNA, there was 25.4% present call for all the available 54,675 probes present on the gene chip. Approximately 17,972 probes were subtracted in this SSH experiment. The results clearly indicated that approximately 50% subtraction was possible in one round of subtraction.

### Example 3

### Predicting Surgical Treatment Response

RNA Reference Pools.

The patients were separated into two groups based on their clinical outcomes.

Group 1: Non-responders for surgical treatment: Lung cancer patients who had recurrence within 5 years after surgical resection.

Group 2: Responders for surgical treatment: Lung cancer patients who did not have recurrence within 5 years after surgical resection.

Two reference RNA pools were prepared for the prediction studies as follows:

Non-responder reference RNA pool: Reference RNA obtained by pooling total RNA obtained from non-responders of surgical treatment.

Responder reference RNA pool: Reference RNA obtained by pooling total RNA obtained from responders of surgical treatment.

The quality of reference RNA was tested using HU 133 plus 2.0 arrays, and the results of scatter plot analyses are shown in Table 2. The results indicated the presence of comparable transcript levels in both reference RNA pools.

TABLE 2

| | |
|---|---|
| Non-Responder | Responder Reference |
| Reference RNA Pool | RNA Pool |

| Quality | Number | Percentage | Number | Percentage |
|---|---|---|---|---|
| Total | 54675 | | 54675 | |
| Probe Sets | | | | |
| Number | 31207 | 57.1% | 30282 | 55.4% |
| Present | | | | |
| Number | 22622 | 41.4% | 23554 | 43.1% |
| Absent | | | | |
| Number | 846 | 1.5% | 839 | 1.5% |
| Marginal | | | | |

Prediction of Recurrence in Stage 1 (Adenocarcinoma) NSCLC Patient:

To predict surgical treatment response, total RNA extracted from a non-responder patient (Patient No. 8T was treated as an unknown patient) was subjected to ribosomal reduction to enrich mRNA species. Approximately, 100 ng mRNA were independently subtracted against the non-responder reference RNA pool and responder reference RNA pool. The subtracted transcripts (unhybridized mRNA) were amplified, and hybridized to human whole genome HG-U133 plus 2.0 GENECHIP (AFFYMETRIX) at 45° C. for 16 hours. After washing and scanning, the data was compared for the SSH efficiency. Prediction results were interpreted based on the present call analysis. Prediction principles and results for patient 8T are shown in FIG. 2.

The results of this analysis indicated that the patient RNA showed lesser present call (18,829) when subtracted against the non-responder reference RNA pool. In contrast, the patient RNA showed more present call (22,013) when subtracted against responder RNA pool. The lower present call of Patient No. 8T when compared to a non-responder reference pool clearly indicated that Patient No. 8T belonged to the non-responder class. The SSH and oligonucleotide microarray data were correlated with clinical outcome.

For use with the instant method, a computer aided design-based microarray data analysis and visualization algorithm was developed. Using this algorithm, it was determined whether recurrence of cancer could be predicted (FIG. 3). Initially, training and testing was conducted with the known samples. Later the algorithm was tested to predict recurrence status of unknown samples (AC 53 and AC331). The algorithm classified the samples exactly to match with their clinical outcome (AC53 and AC 331 as the non-responder patients).

### Example 4

### Predicting Adjuvant therapy Response

Using the instant method and the computer aided design-based microarray data analysis and visualization algorithm, it was determined whether identification of patients who may be benefited from adjuvant therapy response. The patients were separated into two groups based on their clinical outcomes.

Group 1: Non-responders for adjuvant Chemotherapy treatment: Lung cancer patients who died within 10 years after the treatment.

Group 2: Responders for adjuvant Chemotherapy treatment: Lung cancer patients who did not die within 10 years after adjuvant Chemotherapy treatment.

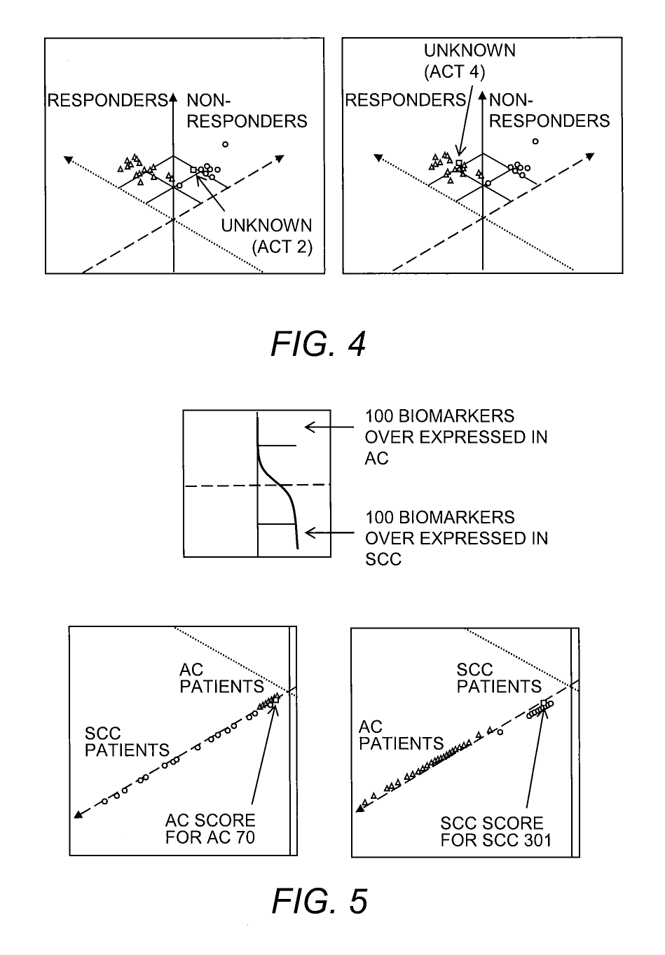

Initially, training and testing was conducted with the known samples. Later, the algorithm was tested to predict adjuvant therapy response of unknown samples (ACT 2 and ACT 4). The algorithm classified the samples exactly to match with their clinical outcome. ACT 2 and ACT 4 were classified as the responder and non-responder, respectively (FIG. 4).

### Example 5

### Predicting Lung Cancer Subclasses

Using the instant method and the computer aided design-based microarray data analysis and visualization algorithm, it was determined whether subclasses of cancer could be determined. RNA from two patients (AC 70 and SCC 301) was subtracted against reference RNA pools from patients with adenocarcinoma (AC) and squamous cell carcinoma (SCC) and oligonucleotides microarray analysis was conducted. Based upon 100 biomarkers found to be overexpressed in adenocarcinoma and squamous cell carcinoma subtypes, AC 70 and SCC 301 were correctly classified as respectively having adenocarcinoma and squamous cell carcinoma (FIG. 5).

What is claimed is:

1. A method for classifying a patient comprising
(a) isolating a total RNA sample from the patient;
(b) subjecting the total RNA to ribosomal RNA reduction, mRNA species enrichment and fragmentation;
(c) subtracting a first aliquot of the fragmented mRNA against a first reference pool of complementary RNA (cRNA);
(d) independently subtracting a second aliquot of the fragmented mRNA against a second reference pool of cRNA;
(e) independently amplifying the subtracted mRNA of (c) and (d) to produce first and second amplified RNAs, respectively;
(f) independently hybridizing the first and second amplified RNAs to oligonucleotide microarrays to generate first and second patterns of hybridization;
(g) comparing the first and second patterns of hybridization to controls to classify the patient.

2. The method of claim 1, wherein the first and second reference pool cRNA are respectively from responders and non-responders to treatment and the patient is classified as a responder or non-responder to treatment.

3. The method of claim 2, wherein the controls comprise oligonucleotides microarray hybridization patterns of responders and non-responders to treatment.

4. The method of claim 2 or 3, wherein the treatment is lung cancer surgery or adjuvant therapy.

5. The method of claim 1, wherein the patient is classified as having a particular class of cancer.

6. The method of claim 5, wherein the cancer is lung cancer.

7. The method of claim 6, wherein the lung cancer is classified as non-small-cell lung carcinoma, small-cell lung carcinoma, cardinoid, or sarcoma.

8. The method of claim 7, wherein the non-small-cell lung carcinoma is subclassified as adenocarcinoma, squamous cell carcinoma or Large Cell carcinoma.

9. The method of claim 8, wherein the first and second reference pool cRNA are respectively from patients with adenocarcinoma and squamous cell carcinoma and the patient is classified as having adenocarcinoma or squamous cell carcinoma.

10. The method of claim 9, wherein the controls comprise oligonucleotides microarray hybridization patterns of patients with adenocarcinoma and squamous cell carcinoma.

11. A kit comprising a first and second reference pool of complementary RNA (cRNA), and one or more oligonucleotides microarrays.

12. The kit of claim 11, wherein the first and second reference pool cRNA are respectively from responders and non-responders to treatment.

13. The kit of claim 12, wherein the treatment is lung cancer surgery or adjuvant therapy.

14. The kit of claim 11, wherein the first and second reference pool cRNA are respectively from patients with adenocarcinoma and squamous cell carcinoma.

15. The kit of claim 11, further comprising computer aided design-based microarray data analysis and visualization software.
